(19) United States
(12) Patent Application Publication
(10) Pub. No.: US 2016/0123949 A1
(43) Pub. Date: May 5, 2016

(54) Measuring Phosphorus in Wastewater Using a Self-Organizing RBF Neural Network

(71) Applicant: Beijing University of Technology, Beijing (CN)

(72) Inventors: Honggui Han, Beijing (CN); Junfei Qiao, Beijing (CN); Wendong Zhou, Beijing (CN)

(73) Assignee: Beijing University of Technology

(21) Appl. No.: 14/620,088

(22) Filed: Feb. 11, 2015

(30) Foreign Application Priority Data
Nov. 2, 2014 (CN) 201410602859.X

(51) Int. Cl. G01N 33/18; G06N 3/08

(52) U.S. Cl. CPC G01N 33/18; G06N 3/08

(57) ABSTRACT

In various implementations, methods and systems are designed for predicting effluent total phosphorus (TP) concentrations in an urban wastewater treatment process (WWTP). To improve efficiency of TP prediction, a particle swarm optimization self-organizing radial basis function (PSO-SORBF) neural network may be established. Implementations may adjust structures and parameters associated with the neural network to train the neural network. The implementations may predict the effluent TP concentrations with reasonably accuracy and allow timely measurement of the effluent TP concentrations. The implementations may further collect online information related to the estimated effluent TP concentrations. This may improve the quality of monitoring processes and enhance management of WWTP.

### CROSS REFERENCE TO RELATED PATENT APPLICATIONS

This application claims priority to Chinese Patent Application No. 201410602859.X, filed on Nov. 2, 2014, entitled “a Soft-Computing Method for the Effluent Total Phosphorus Based on a Self-Organizing PSO-RBF Neural Network,” which is hereby incorporated by reference in its entirety.

### TECHNICAL FIELD

Implementations here related to control environment engineering, more specifically related to methods and systems for determining effluent total phosphorus (TP) concentrations in the urban wastewater treatment process (WWTP).

### BACKGROUND

Biogeochemical characteristics of phosphorus play a significant role in eutrophication processes. Phosphorus may accumulate in lake sediments during heavy loading periods and release from sediments into the overlying water after the external loading is reduced. The released phosphorus sustains the eutrophication processes and cycles between overlying water and sediments through algal growth, organic deposition, decomposition and release. Therefore, phosphorus is generally recognized as the limiting factor in the process of eutrophication. Restoration efforts to control phosphorus from WWTP into rivers are considered to be important strategies for decreasing cyanobacterial risks in the environment.

To reduce levels of phosphate, some design principles and various mechanisms are recently adopted to produce low effluent TP concentrations in urban WWTP. The effluent TP concentration is an index of water qualities in the urban WWTP. However, using conventional technologies, it is difficult to timely estimate the effluent TP concentration under closed loop control. The timely and/or online detection technology of effluent TP concentrations is a bottleneck for the control of the urban WWTP. Moreover, the real-time information of effluent TP concentrations can enhance the quality monitoring level and alleviate the current situation of wastewater to strengthen the whole management of WWTP. Therefore, the timely detection of effluent TP concentration owns both great economic benefit and environmental benefit.

Methods for monitoring the effluent TP concentration may include: spectrophotometry method, gas chromatography method, liquid chromatography method, electrode method, and mechanism model. However, the spectrophotometry method, gas chromatography method, liquid chromatography method and electrode method rely upon previously collected data analysis of primary variables. Some of the variables, such as gas chromatography method, require more than 30 minutes to obtain. This makes these approaches inadequate for real-time and/or online monitoring. The mechanism model studies the phosphorus dynamics to obtain the effluent TP concentration online based on the biogeochemical characteristics of phosphorus. However, significant errors may be incurred in the measurement of effluent TP concentrations. Moreover, because of the different conditions of every urban WWTP, a common model is difficult to be determined. Thus, technologies for timely monitoring effluent TP concentrations are not well developed.

### SUMMARY

Methods and systems are designed for effluent TP concentrations based on a PSO-SORBF neural network in various implementations. In various implements, the inputs are those variables that are easy to measure and the outputs are estimates of the effluent TP concentration. Since the input-output relationship is encoded in the data used to calibrate the model, a method is used to reconstruct it and then to estimate the output variables. In general, the procedure of soft-computing method comprise three parts: data acquisition, data pre-processing and model design. For various implementations, an experimental hardware is set up. The historical process data are routinely acquired and stored in the data acquisition system. The data may be easily retrieved in the method. The variables whose data are easy to measure by the instruments comprise: influent TP, oxidation-reduction potential (ORP) in the anaerobic tank, dissolved oxygen (DO) concentration in the aerobic tank, temperature in the aerobic tank, total suspended solids (TSS) in the aerobic tank, effluent pH, chemical oxygen demand (COD) concentration in the aerobic tank and total nutrients (TN) concentration in the aerobic tank. Then, data pre-processing and model design are developed to predict the effluent TP concentrations.

Various implementations adopts the following technical scheme and implementation steps:

A soft-computing method for the effluent TP concentration based on a PSO-SORBF neural network, its characteristic and steps include the following steps: (1) Selecting input variables, (2) Initializing the PSO-SORBF neural network, (3) training the PSO-SORBF neural network, and (4) setting the PSO-SORBF neural network.

### (1) Select Input Variables

Remarkable characteristics of the data acquired in urban WWTP are redundancy and possibly insignificance. And the choice of the input variables that influence the model output is a crucial stage. Therefore, it is necessary to select the suitable input variables and prepare their data before using the soft-computing method. Moreover, variable selection comprise choosing those easy to measure variables that are most informative for the process being modelled, as well as those that provide the highest generalization ability. In various implementations, the partial least squares (PLS) method is used to extract the input variables for the soft-computing method.

In various implementations, a history data set {X, y} is used for the variable selection. Since the variables acquired from experimental hardware are influent TP, ORP, DO, temperature, TSS, effluent pH, COD and TN. X is an n×8 process variable matrix, and y is the dependent n×1 variable vector. The PLS method can model both outer and inner relations between X and y. For the PLS method, X and y may be described as:

$$X = {TP}^{T} + E = {\sum }_{i = 1}^{8} {t}_{i} {p}_{i}^{T} + E , \\ y = {UQ}^{T} + F = {\sum }_{i = 1}^{8} {u}_{i} {q}_{i}^{T} + F , \qquad ( 1 )$$

where T, P and E are the score matrix, loading matrix and residual matrix of X, respectively. U, Q and F are the score matrix, loading matrix and residual matrix of y. tᵢ, pᵢ, uᵢ and qᵢ are the vectors of T, P, U and Q. In addition, the inner relationship between X and y is shown as follow:

ûᵢ=bᵢtᵢ,

bᵢ=uᵢ^{T}tᵢ/tᵢ^{T}t_{l ,} (2)

where i=1, 2, . . . ,8, bᵢ is the regression coefficients between the tᵢ from X and uᵢ from y. Then, the cross-validation values for the components in X and y are described as:

$${R}_{i} = {G}_{i} / G , i = 1 , 2 , … , 8 ; \\ G = {\sum }_{i = 1}^{8} ‖ {\hat{u}}_{i} - {t}_{i} ‖ , \\ {G}_{i} = ‖ {\hat{u}}_{i} - {t}_{i} ‖ , \qquad ( 3 )$$

if Rᵢ<ξ, ξ∈(0, 0.1), the ith component is the right input variable for the soft-computing model. Based on the PLS method, the selected input variables are influent TP, ORP, DO, T, TSS and effluent pH in various implementations.

### (2) Initialize the PSO-SORBF Neural Network

The initial structure of PSO-SORBF neural network comprise three layers: input layer, hidden layer and output layer. There are 6 neurons in the input layer, K neurons in the hidden layer and 1 neuron in the output layer, K>2 is a positive integer. The number of training samples is T. The input vector of PSO-SORBF neural network is x(t)=[x₁(t), x₂(t), x₃(t), x₄(t), x₅(t), x₆(t)] at time t. x₁(t) is the value of influent TP, x₂(t) is the value of ORP, x₃(t) is the value of DO, x₄(t) is the value of temperature, x₅(t) is the value of TSS, and x₆(t) is the value of effluent pH at time t respectively. y(t) is the output of PSO-SORBF neural network, and y_{d}(t) is the real value of effluent TP concentration at time t respectively. The output of PSO-SORBF neural network may be described:

$$y ( t ) = {\sum }_{k = 1}^{K} {w}_{k} ( t ) {φ}_{k} ( x ( t ) ) , \qquad ( 4 )$$

where wₖ is the output weight between the kth hidden neuron and the output neuron, k=1, 2, . . . , K, K is the number of hidden neurons, and φₖ is the RBF of kth hidden neuron which is usually defined by a normalized Gaussian function:

φₖ(x(t))=e^{(-∥x(t)-μ}^{ₖ}^{(t)∥}^{²}^{/2σ}^{ₖ}^{²}^{(t))}, (5)

μₖ=[μ_{k,1}, μ_{k,2}, . . . , μ_{k,6}] denotes the center vector of the kth hidden neuron, σₖ is the width of the kth hidden neuron, ∥x(t)−μₖ(t)∥ is the Euclidean distance between x(t) and μₖ(t).

### (3) Train the PSO-SORBF Neural Network

\textcircled{1} Initialize the acceleration constants c₁ and c₂, c₁∈(0, 1), c₂∈(0, 1), and the balance factor α∈[0, 1]. During the particle initialization stage, let the position of the ith particle in the searching space be represented as:

aᵢ=[u_{i,1}, σ_{i,1}, w_{i,1}, μ_{i,2}, σ_{i,2}, w_{i,2} . . . μ_{i,K}, σ_{i,K}, w_{i,K}], (6)

where aᵢ is the position of ith particle, i=1, 2, . . . , s, and s is the total number of particles, s>2 is a positive integer. μ_{i,k}=[μ_{i,k,1}, μ_{i,k,2}, . . . , μ_{i,k,6}], σ_{i,k}, w_{i,k} are the center, width and output weight of the kth hidden neuron in the ith particle, and the initial values are ∥μ_{i,k}∥<1, σ_{i,k}∈(0, 1), w_{i,k}∈(0, 1). Kᵢ is the number of hidden neurons in the ith particle. Simultaneously, initialize the velocity of particle:

vᵢ=[v_{i,1}, v_{i,2}, . . . v_{i,D}_{ᵢ}], (7)

where vᵢ is velocity of ith particle, Dᵢ is the dimension of the ith particle, and Dᵢ=3Kᵢ.

\textcircled{2} From the input of neural network x(t) and the dimensions Dᵢ of each particle, the fitness value of each particle may be calculated:

$$\begin{matrix}f ( {a}_{i} ( t ) ) = {E}_{i} ( t ) + α {K}_{i} ( t ) , & ( 8 ) \\ where & \\ {E}_{i} ( t ) = \sqrt{\frac{1}{2 T} {\sum }_{t = 1}^{T} {( y ( t ) - {y}_{d} ( t ) )}^{2}} , & ( 9 )\end{matrix}$$

i=1, 2, . . . , s, Kᵢ(t) is the number of hidden neurons in the ith particle at time t, T is the number of the training samples.

\textcircled{3} Calculate the inertia weight of each particle:

ωᵢ(t)=γ(t)Aᵢ(t), (10)

where ωᵢ(t) is the inertia weight of the ith particle at time t, and

γ(t)=(C−S(t)/1000)^{-t},

S(t)=fₘᵢₙ(a(t))/fₘₐₓ(a(t)),

Aᵢ(t)=f(g(t))/f(aᵢ(t)), (11)

C is a constant, and C∈[1, 5], fₘᵢₙ(a(t)), fₘₐₓ(a(t)) are the minimum fitness value and the maximum fitness value at time t, and g(t)=[g₁(t), g₂(t), . . . , g_{D}(t)] is the global best position, fₘᵢₙ(a(t)), fₘₐₓ(a(t)) and g(t) may be expressed as:

$$\{ \begin{matrix}{f}_{min} ( a ( t ) ) = Min ( f ( {a}_{i} ( t ) ) ) \\ {f}_{max} ( a ( t ) ) = Max ( f ( {a}_{i} ( t ) ) )\end{matrix} , \\ g ( t ) = {argmin}_{{p}_{i}} ( f ( {p}_{i} ( t ) ) ) , 1 \leq i \leq s , \qquad ( 12 )$$

where pᵢ(t)=[p_{i,1}(t), p_{i,2}(t), . . . , p_{i,D}(t)] is the best position of the ith particle:

$${p}_{i} ( t + 1 ) = \{ \begin{matrix}{p}_{i} ( t ) , & if f ( {a}_{i} ( t + 1 ) ) \geq f ( {p}_{i} ( t ) ) \\ {a}_{i} ( t + 1 ) , & otherwise\end{matrix} . \qquad ( 13 )$$

\textcircled{4} Update the position and velocity of each particle:

$${v}_{i , d} ( t + 1 ) = ω {v}_{i , d} ( t ) + {c}_{1} {r}_{1} ( {p}_{i , d} ( t ) - {a}_{i , d} ( t ) ) + {c}_{2} {r}_{2} ( {g}_{d} ( t ) - {a}_{i , d} ( t ) ) , \\ g ( t ) = {arg min}_{{p}_{i}} ( f ( {p}_{i} ( t ) ) ) , 1 \leq i \leq s , \qquad ( 14 )$$

where r₁ and r₂ are the coefficient of the particle and global best position respectively, r₁∈[0, 1] and r₂∈[0, 1].

\textcircled{5} Search the best number of hidden neurons K_{best} according to the global best position g(t), and update the number of hidden neurons in the particles:

$${K}_{i} = \{ \begin{matrix}{K}_{i} - 1 & if ( {K}_{best} < {K}_{i} ) \\ {K}_{i} + 1 & if ( {K}_{best} \geq {K}_{i} )\end{matrix} . \qquad ( 15 )$$

\textcircled{6} Import the training sample x(t+1), and repeat the steps \textcircled{2}-\textcircled{5}, then, stop the training process after all of the training samples are imported to the neural network.

(4) The Testing Samples are then Set to the Trained PSO-SORBF Neural Network.

The outputs of PSO-SORBF neural network is the predicting values of effluent TP concentration. Moreover, the program of this soft-computing method has been designed based on the former analysis. The program environment of the proposed soft-computing method comprise a Windows 8 64-bit operating system, a clock speed of 2.6 GHz and 4 GB of RAM. And the program is based on the Matlab 2010 under the operating system.

In some implementations, in order to detect the effluent TP concentration online and with acceptable accuracy, a method is developed in various implementations. The results demonstrate that the effluent TP trends in WWTP may be predicted with acceptable accuracy using the influent TP, ORP, DO, temperature, TSS, and effluent pH data as input variables. This soft-computing method can predict the effluent TP concentration with acceptable accuracy and solve the problem that the effluent TP concentration is difficult to be measured online.

This method is based on the PSO-SORBF neural network in various implementations, which is able to optimize both the parameters and the network size during the learning process simultaneously. The advantages of the proposed PSO-SORBF neural network are that it can simplify and accelerate the structure optimization process of the RBF neural network, and can predict the effluent TP concentration accurately. Moreover, the predicting performance shows that the PSO-SORBF neural network-based soft-computing method can match system nonlinear dynamics. Therefore, this soft-computing method performs well in the whole operating space.

Various implementations utilizes six input variables in this soft-computing method to predict the effluent TP concentration. In fact, it is in the scope of various implementations that any of the variables: the influent TP, ORP, DO, temperature, TSS, effluent pH, COD and TN, are used to predict the effluent TP concentration. Moreover, this soft-computing method is also able to predict the others variables in urban WWTP.

### BRIEF DESCRIPTION OF THE DRAWINGS

The detailed description is described with reference to the accompanying figures.

FIG. 1 shows the overall flow chart of a method for predicting effluent TP concentration in various implementations.

FIG. 2 shows the structure of PSO-SORBF neural network in various implementations.

FIG. 3 shows training results of implementations.

FIG. 4 shows training errors of implementations.

FIG. 5 shows predicting results of implementations.

FIG. 6 shows the predicting error of implementations.

FIGS. 7-18 show tables 1-16 including experimental data of various implementations.

### DETAILED DESCRIPTION

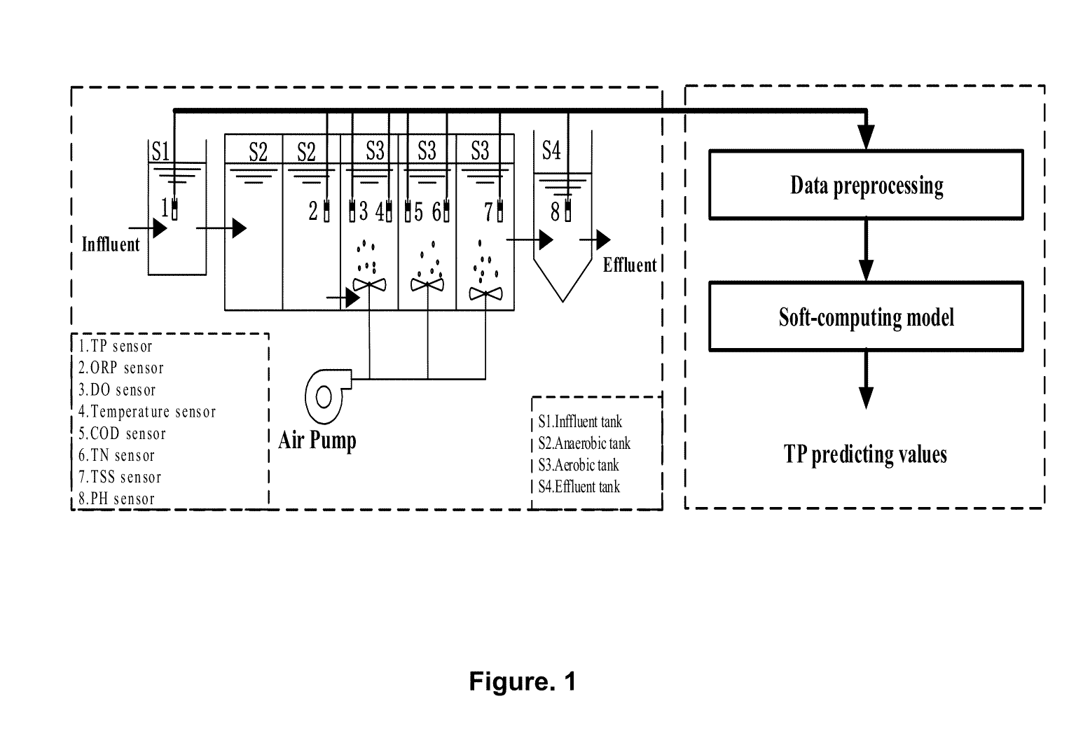

Various implementations of methods and systems are developed to predict the effluent TP concentration based on a PSO-SORBF neural network in various implementations. For the implementations, inputs of the neural network are variables that are easy to measure and outputs of the neural network are estimates of the effluent TP concentration. In general, the procedure of soft-computing method comprises three parts: data acquisition, data pre-processing and model design. For various implementations, an experimental hardware is set up as shown in FIG. 1. The historical process data are routinely acquired and stored in the data acquisition system. The data may be easily retrieved. The variables whose data are easy to measure by the instruments comprise: influent TP, ORP in the anaerobic tank, DO concentration in the aerobic tank, temperature in the aerobic tank, TSS in the aerobic tank, effluent pH, COD concentration in the aerobic tank and TN concentration in the aerobic tank. Then, data pre-processing and model design are developed to predict the effluent TP concentration.

Various implementations adopts the following technical scheme and implementation steps for the effluent TP concentration based on a PSO-SORBF neural network. The characteristic and steps are described as follow.

### (1) Select Input Variables

Remarkable characteristics of the data acquired in urban WWTP are redundancy and possibly insignificance. And the choice of the input variables that influence the model output is a crucial stage. Therefore, it is necessary to select the suitable input variables and prepare their data before using the soft-computing method. Moreover, variable selection comprises choosing those easy to measure variables that are most informative for the process being modelled, as well as those that provide the highest generalization ability. In various implementations, the PLS method is used to extract the input variables for the soft-computing method.

The experimental data is obtained from an urban WWTP in 2014. There are 245 groups of samples which are divided into two parts: 165 groups of training samples and 80 groups of testing samples.

In various implementations, a history data set {X, y} is used for variable selection. Since the variables acquired from experimental hardware are influent TP, ORP, DO, temperature, TSS, effluent pH, COD and TN. X is a 165×8 process variable matrix, and y is the dependent 165×1 variable vector. The PLS method can model both outer and inner relations between X and y. For the PLS method, X and y may be described as follows:

$$X = T {P}^{T} + E = {\sum }_{i = 1}^{8} {t}_{i} {p}_{i}^{T} + E , \\ y = U {Q}^{T} + F = {\sum }_{i = 1}^{8} {u}_{i} {q}_{i}^{T} + F , \qquad ( 16 )$$

where T, P and E are the score matrix, loading matrix and residual matrix of X, respectively. U, Q and F are the score matrix, loading matrix and residual matrix of y. tᵢ, pᵢ, uᵢ and qᵢ are the vectors of T, P, U and Q. In addition, the inner relationship between X and y is shown as follow:

ûᵢ=bᵢtᵢ,

bᵢ=uᵢ^{T}tᵢ/tᵢ^{T}tᵢ, (17)

where i=1, 2, . . . , 8, bᵢ is the regression coefficients between the tᵢ from X and uᵢ from y. Then, the cross-validation values for the components in X and y are described as:

$${R}_{i} = {G}_{i} / G , i = 1 , 2 , … , 8 ; \\ G = {\sum }_{i = 1}^{8} ‖ {\hat{u}}_{i} - {t}_{i} ‖ , \\ {G}_{i} = ‖ {\hat{u}}_{i} - {t}_{i} ‖ , \qquad ( 18 )$$

if Rᵢ<ξ, ξ=0.01, the ith component is the right input variable for the soft-computing model. Based on the PLS method, the selected input variables are influent TP, ORP, DO, T, TSS and effluent pH in various implementations.

### (2) Initialize the PSO-SORBF Neural Network

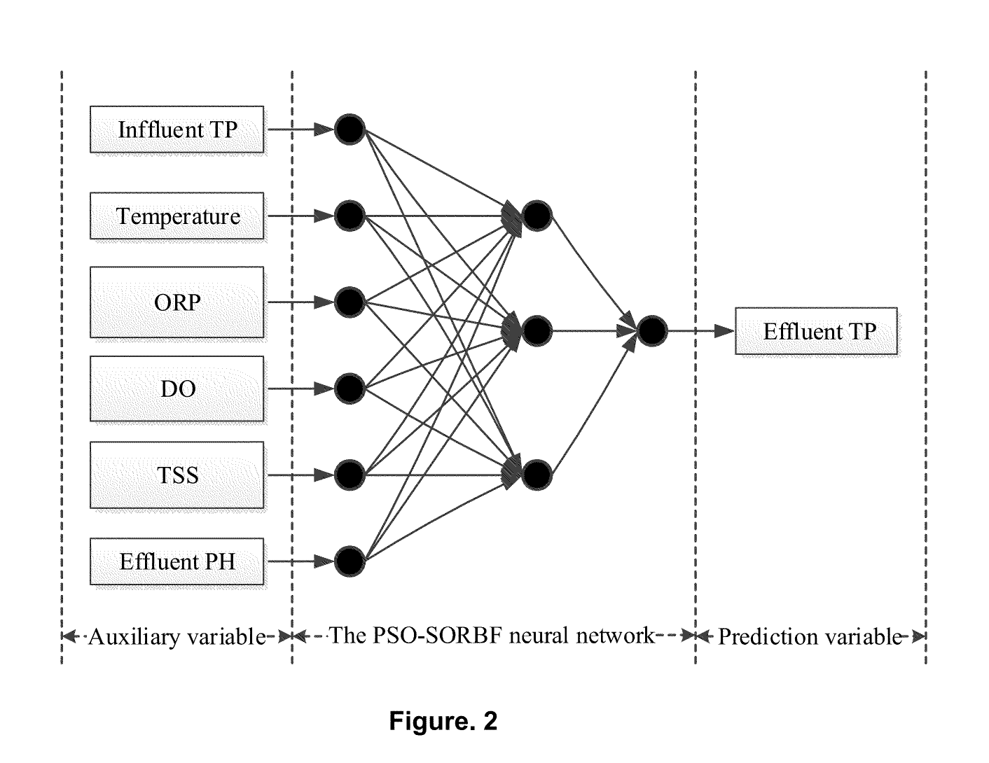

The initial structure of PSO-SORBF neural network, which is shown in FIG. 2 comprises three layers: input layer, hidden layer and output layer. There are 6 neurons in the input layer, K neurons in the hidden layer and 1 neuron in the output layer, K=3. The number of training samples is T. The input vector of PSO-SORBF neural network is x(t)=[x₁(t), x₂(t), x₃(t), x₄(t), x₅(t), x₆(t)] at time t. x₁(t) is the value of influent TP, x₂(t) is the value of ORP, x₃(t) is the value of DO, x₄(t) is the value of temperature, x₅(t) is the value of TSS, and x₆(t) is the value of effluent pH at time t respectively. y(t) is the output of PSO-SORBF neural network, and y_{d}(t) is the real value of effluent TP concentration at time t respectively. The output of PSO-SORBF neural network may be described as:

$$y ( t ) = {\sum }_{k = 1}^{K} {w}_{k} ( t ) {φ}_{k} ( x ( t ) ) , \qquad ( 19 )$$

where wₖ is the output weight between the kth hidden neuron and the output neuron, k=1, 2, . . . , K, K is the number of hidden neurons, and φₖ is the RBF of kth hidden neuron which is usually defined by a normalized Gaussian function:

φₖ(x(t))=e^{(-∥a(t)-μ}^{ₖ}^{(i)∥}^{²}^{/2σ}^{ₖ}^{²}^{(t))}, (20)

μₖ denotes the center vector of the kth hidden neuron, σₖ is the width of the kth hidden neuron, ∥x(t)−μₖ(t)∥ is the Euclidean distance between x(t) and μₖ(t).

### (3) Train the PSO-SORBF Neural Network

\textcircled{1} Initialize the acceleration constants c₁ and c₂, c₁=0.4, c₂=0.6, and the balance factor α=0.1. During the particle initialization stage, let the position of the ith particle in the searching space be represented as:

aᵢ=[μ_{i,1}, σ_{i,1}, w_{i,1}, μ_{i,2}, σ_{i,2}, w_{i,2} . . . μ_{i,K}_{_{l , σ}}_{i,K}, w_{i,K}_{ᵢ}], (21)

where aᵢ is the position of ith particle, i=1, 2, . . . , s, and s is the total number of particles, s=3 is a positive integer. μ_{i,k}, σ_{i,k}, w_{i,k} are the center, width and output weight of the kth hidden neuron in the ith particle, and the initial values of the center, width and output weight are randomly generated within (0, 1). K₁=2, K₂=3, K₃=4. Initialize the velocity of particle:

vᵢ=[v_{i,1}, v_{i,2}, . . . v_{i,D}], (22)

where vᵢ is velocity of ith particle, Dᵢ is the dimension of the ith particle, and Dᵢ=3Kᵢ.

\textcircled{2} From the input of neural network x(t) and the dimensions Dᵢ of each particle, the fitness value of each particle may be calculated:

$$\begin{matrix}f ( {a}_{i} ( t ) ) = {E}_{i} ( t ) + α {K}_{i} ( t ) , & ( 23 ) \\ where & \\ {E}_{i} ( t ) = \sqrt{\frac{1}{2 T} {\sum }_{t = 1}^{T} {( y ( t ) - {y}_{d} ( t ) )}^{2}} , & ( 24 )\end{matrix}$$

i=1, 2, . . . , s, Kᵢ(t) is the number of hidden neurons in the ith particle at time t, T is the number of the training samples.

\hat{3} Calculate the inertia weight of each particle:

ωᵢ(t)=γ(t)Aᵢ(t), (25)

where ωᵢ(t) is the inertia weight of the ith particle at time t, and

γ(t)=(C−S(t)/1000)^{-t},

S(t)=fₘᵢₙ(a(t))/fₘₐₓ(a(t)),

Aᵢ(t)=f(g(t))/f(aᵢ(t)), (26)

C=2, fₘᵢₙ(a(t)), fₘₐₓ(a(t)) are the minimum fitness value and the maximum fitness value, and g(t)=[g₁(t), g₂(t), . . . , g_{D}(t)] is the global best position, fₘᵢₙ(a(t)), fₘₐₓ(a(t)) and g(t) may be expressed as:

$$\{ \begin{matrix}{f}_{min} ( a ( t ) ) = Min ( f ( {a}_{i} ( t ) ) ) \\ {f}_{max} ( a ( t ) ) = Max ( f ( {a}_{i} ( t ) ) )\end{matrix} , \\ g ( t ) = {arg min}_{{p}_{i}} ( f ( {p}_{i} ( t ) ) ) , 1 \leq i \leq s , \qquad ( 27 )$$

where pᵢ(t)=[p_{i,1}(t), p_{i,2}(t), . . . , p_{i,D}(t)] is the best position of the ith particle:

$${p}_{i} ( t + 1 ) = \{ \begin{matrix}{p}_{i} ( t ) , & if & f ( {a}_{i} ( t + 1 ) ) \geq f ( {p}_{i} ( t ) ) \\ {a}_{i} ( t + 1 ) , & & otherwise\end{matrix} . \qquad ( 28 )$$

\textcircled{4} Update the position and velocity of each particle:

$${v}_{i , d} ( t + 1 ) = ω {v}_{i , d} ( t ) + {c}_{1} {r}_{1} ( {p}_{i , d} ( t ) - {a}_{i , d} ( t ) ) + {c}_{2} {r}_{2} ( {g}_{d} ( t ) - {a}_{i , d} ( t ) ) , \\ g ( t ) = {arg min}_{{p}_{i}} ( f ( {p}_{i} ( t ) ) ) , 1 \leq i \leq s , \qquad ( 29 )$$

where r₁ and r₂ are the coefficient of the particle and global best position respectively, r₁=0.75 and r₂=0.90.

\textcircled{5} Search the best number of hidden neurons K_{best} according to the global best position g(t), and update the number of hidden neurons in the particles:

$${K}_{i} = \{ \begin{matrix}{K}_{i} - 1 & if ( {K}_{best} < {K}_{i} ) \\ {K}_{i} + 1 & if ( {K}_{best} \geq {K}_{i} )\end{matrix} . \qquad ( 30 )$$

\textcircled{6} Import the training sample x(t+1), and repeat the steps \textcircled{2}-\textcircled{5}, then, stop the training process after all of the training samples are imported to the neural network.

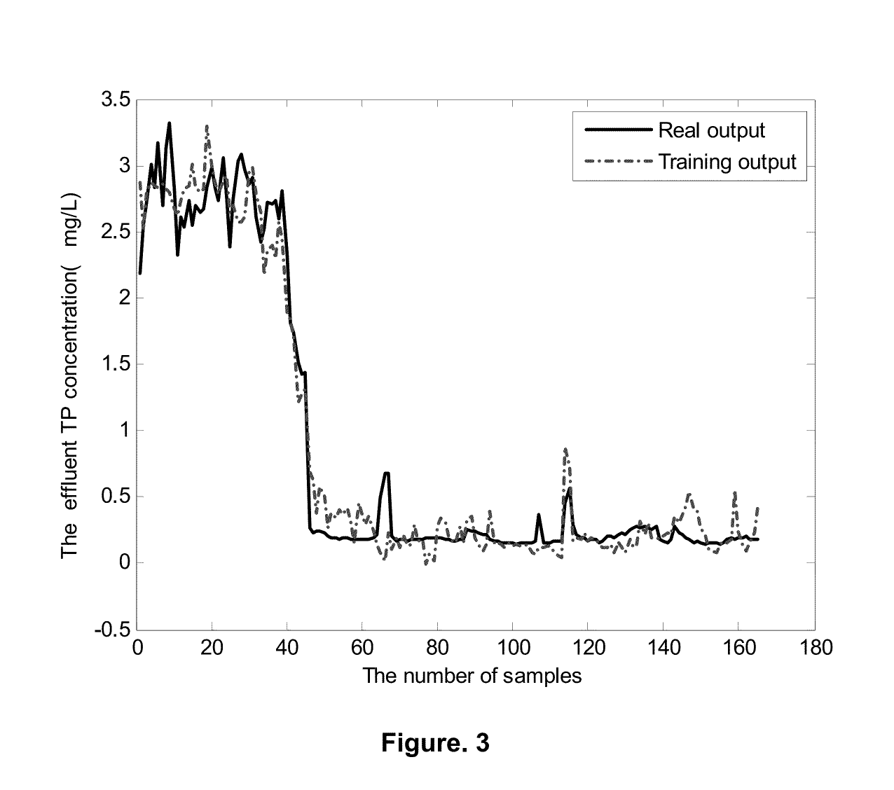

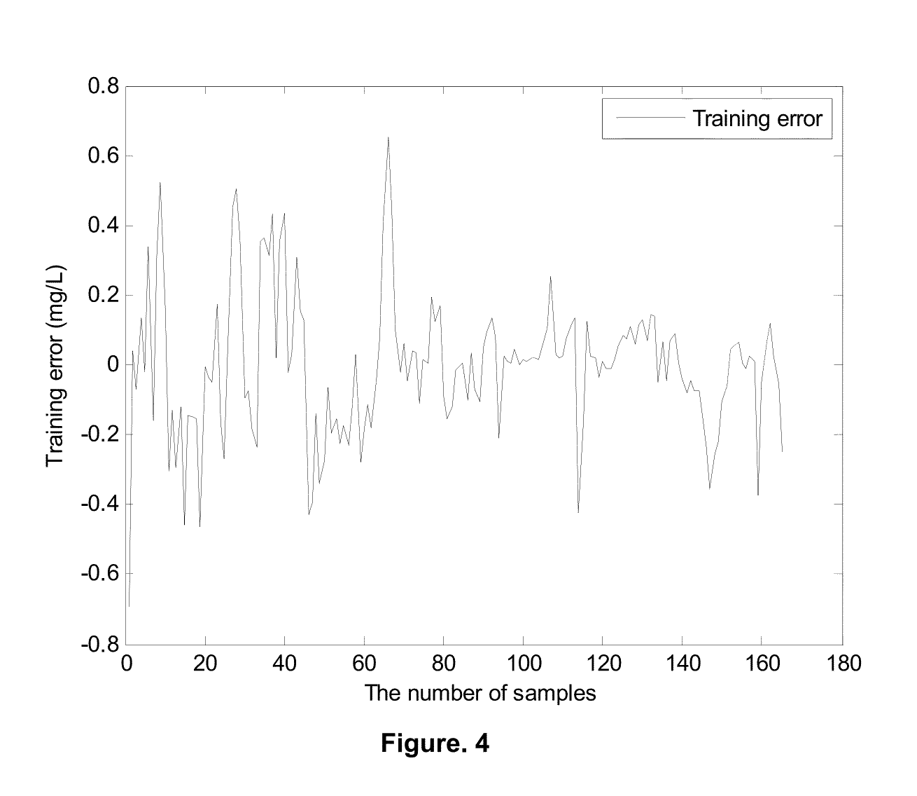

The training results of the soft-computing method are shown in FIG. 3. X axis shows the number of samples. Y axis shows the effluent TP concentration. The unit of Y axis is mg/L. The solid line presents the real values of effluent TP concentration. The dotted line shows the outputs of soft-computing method in the training process. The errors between the real values and the outputs of soft-computing method in the training process are shown in FIG. 4. X axis shows the number of samples. Y axis shows the training error. The unit of Y axis is mg/L.

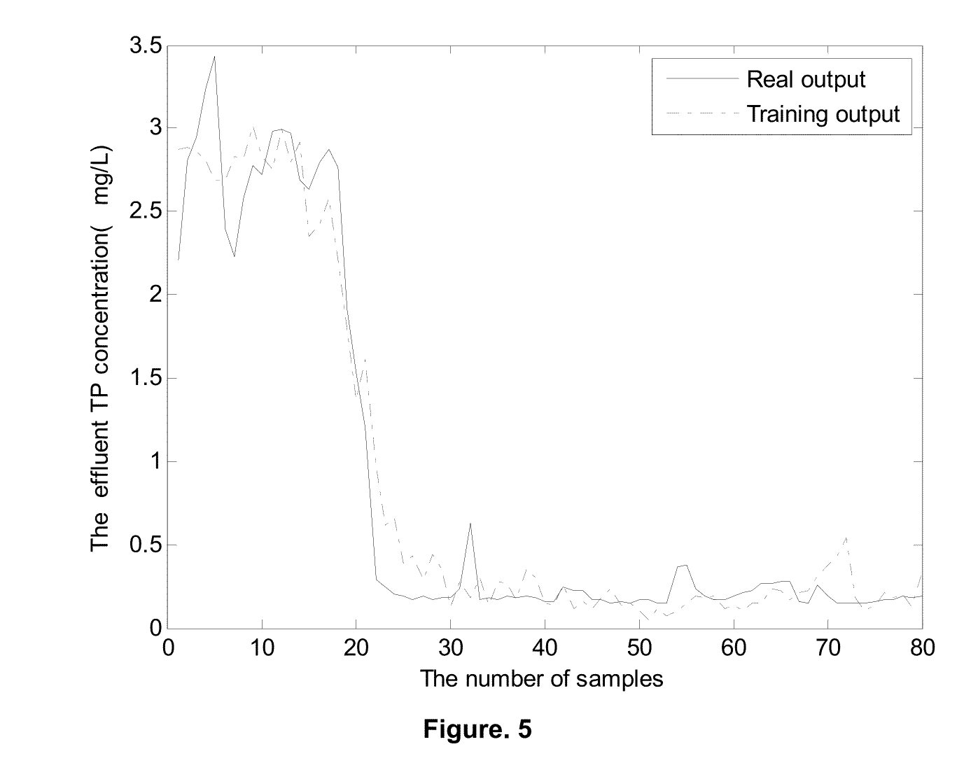

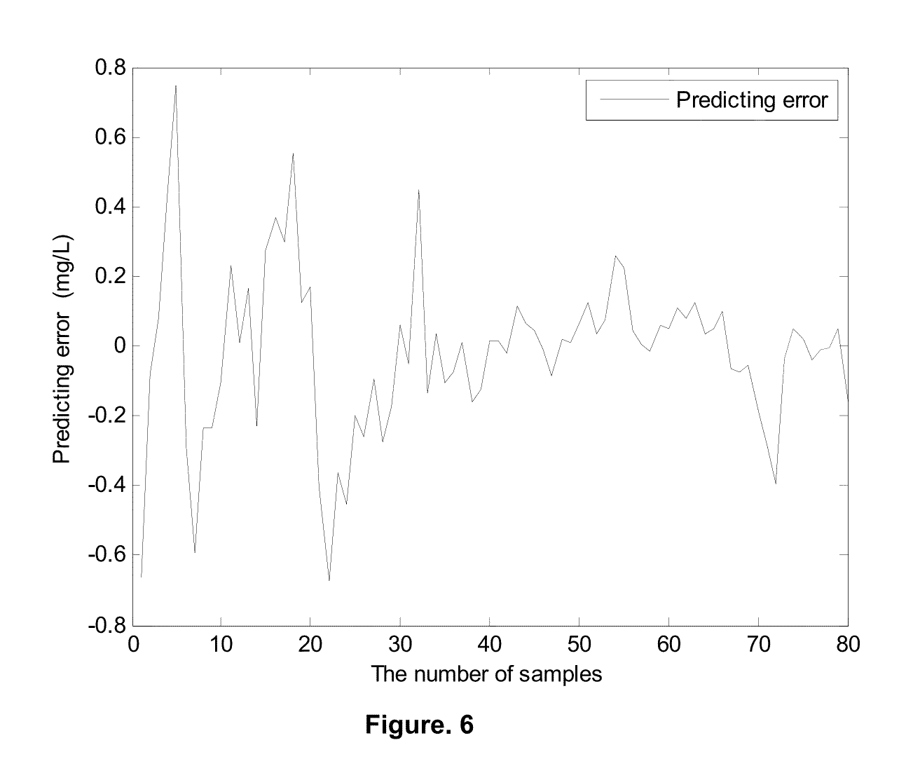

(4) The testing samples are then set to the trained PSO-SORBF neural network. The outputs of the PSO-SORBF neural network are the predicting values of effluent TP concentration. The predicting results are shown in FIG. 5. X axis shows the number of samples. Y axis shows the effluent TP concentration. The unit of Y axis is mg/L. The solid line presents the real values of effluent TP concentration. The dotted line shows the outputs of soft-computing method in the testing process. The errors between the real values and the outputs of soft-computing method in the testing process are shown in FIG. 6. X axis shows the number of samples. Y axis shows the training error. The unit of Y axis is mg/L.

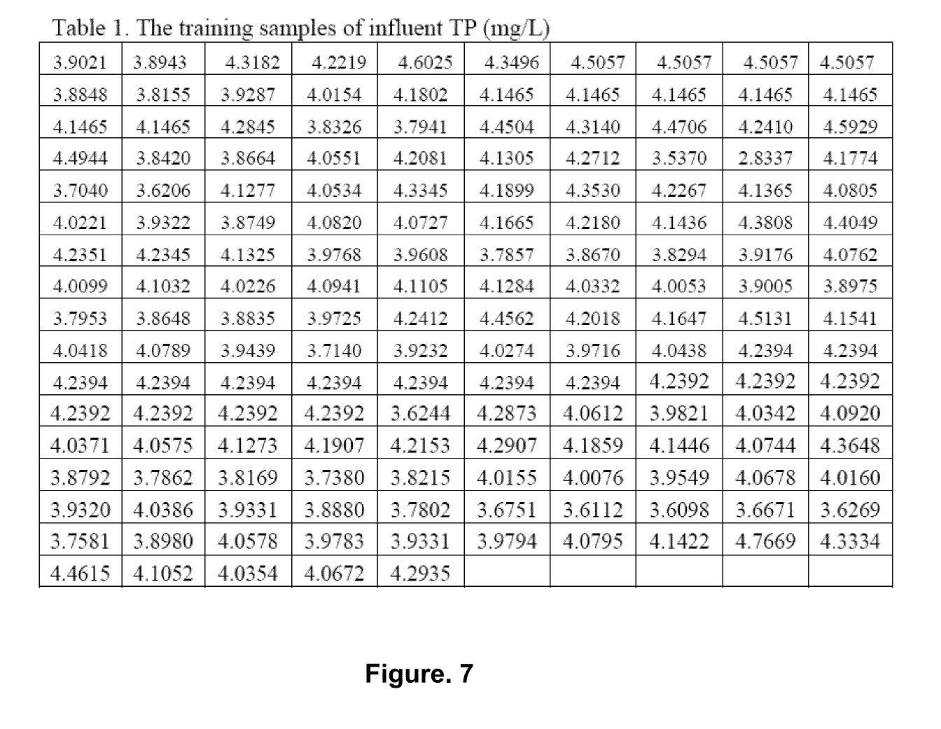

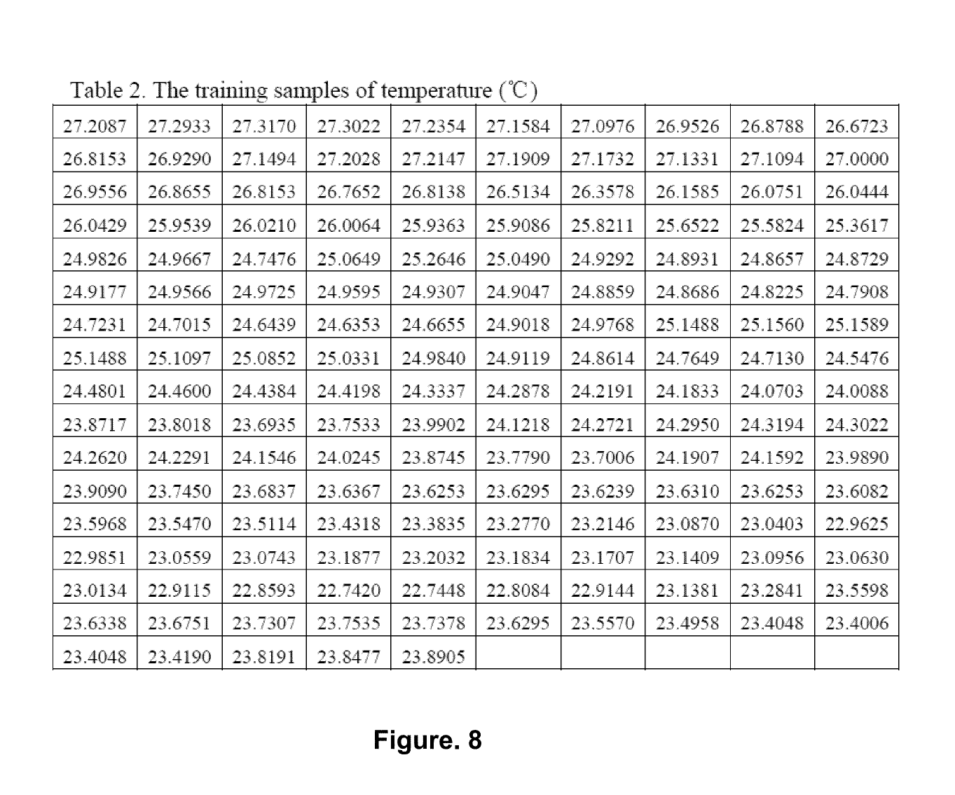

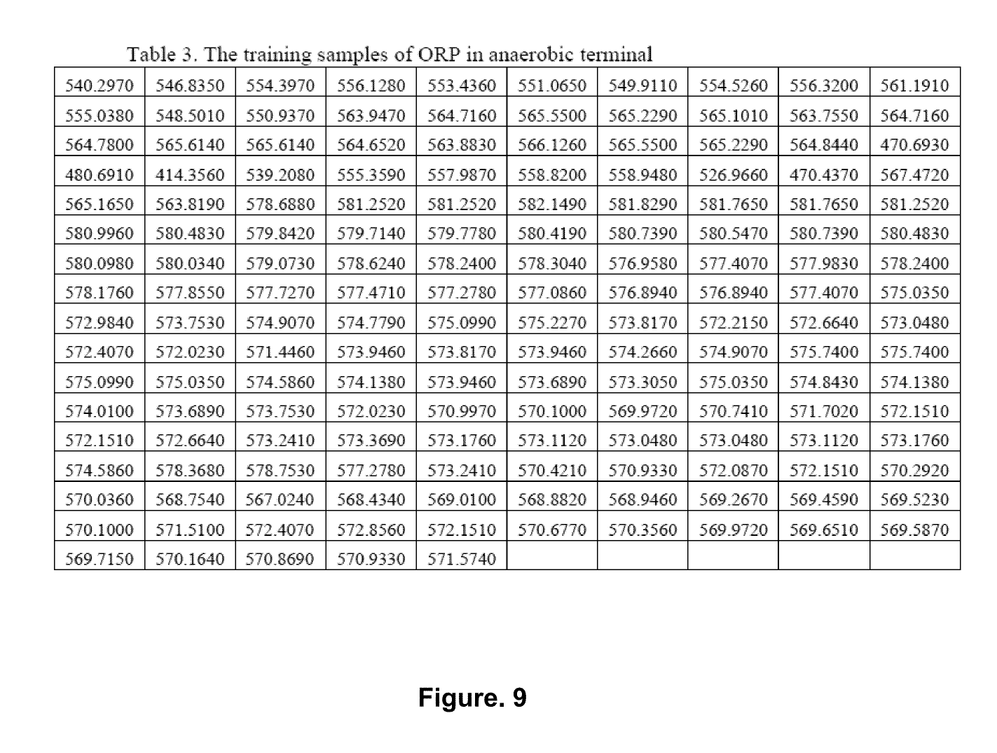

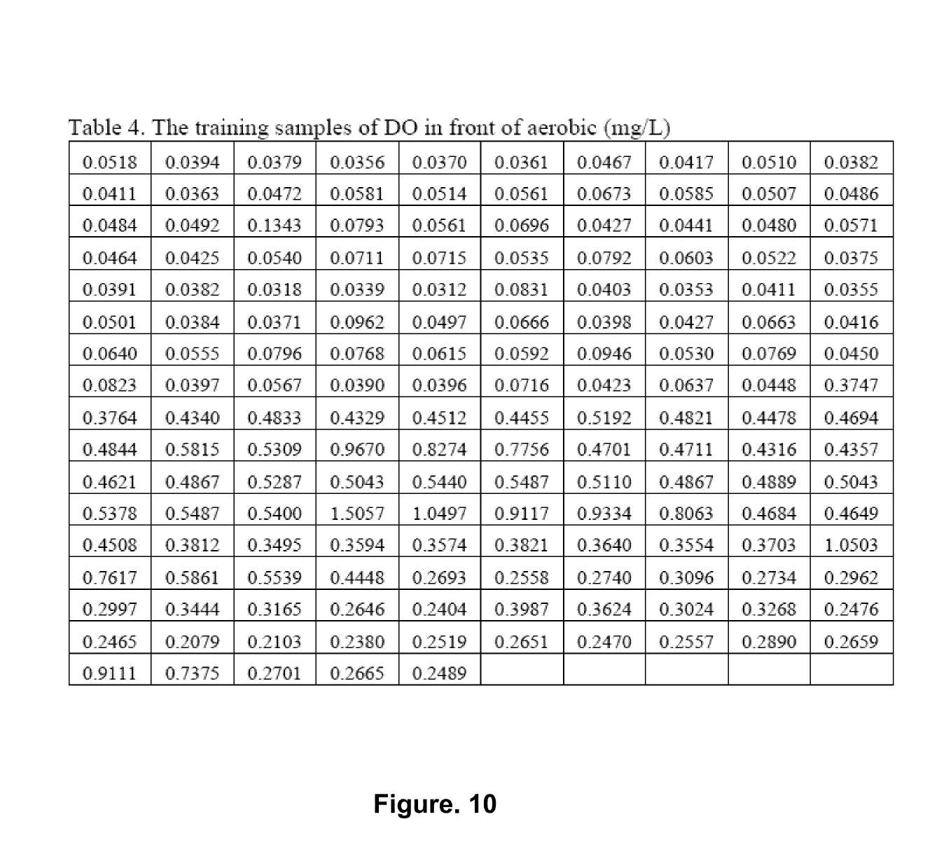

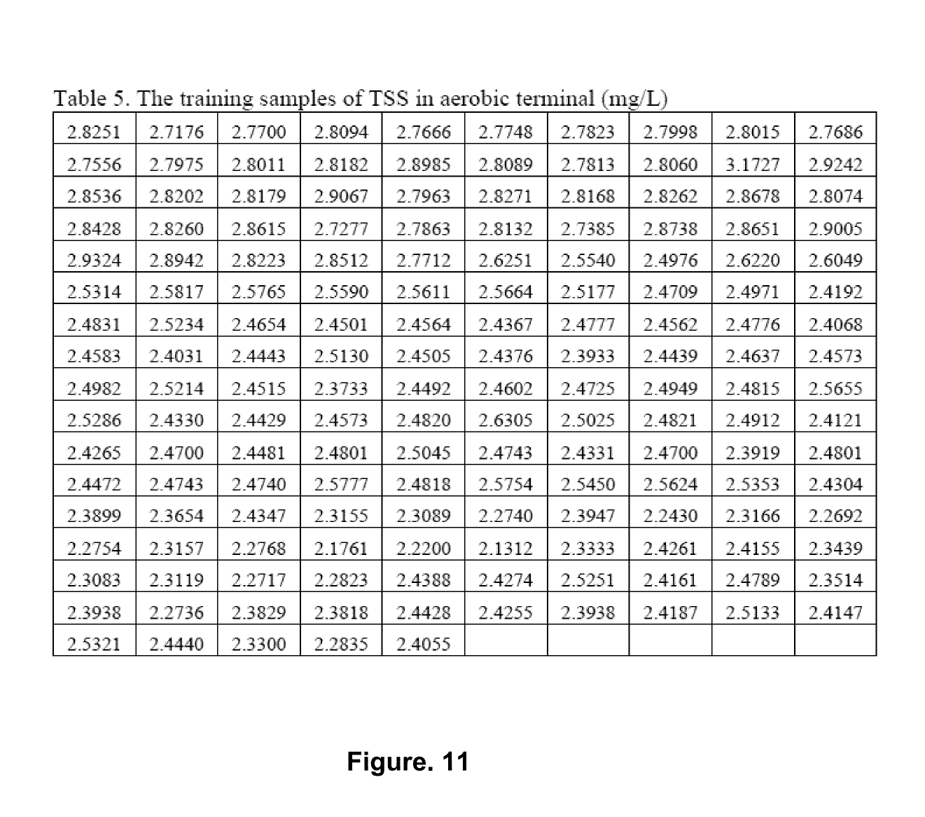

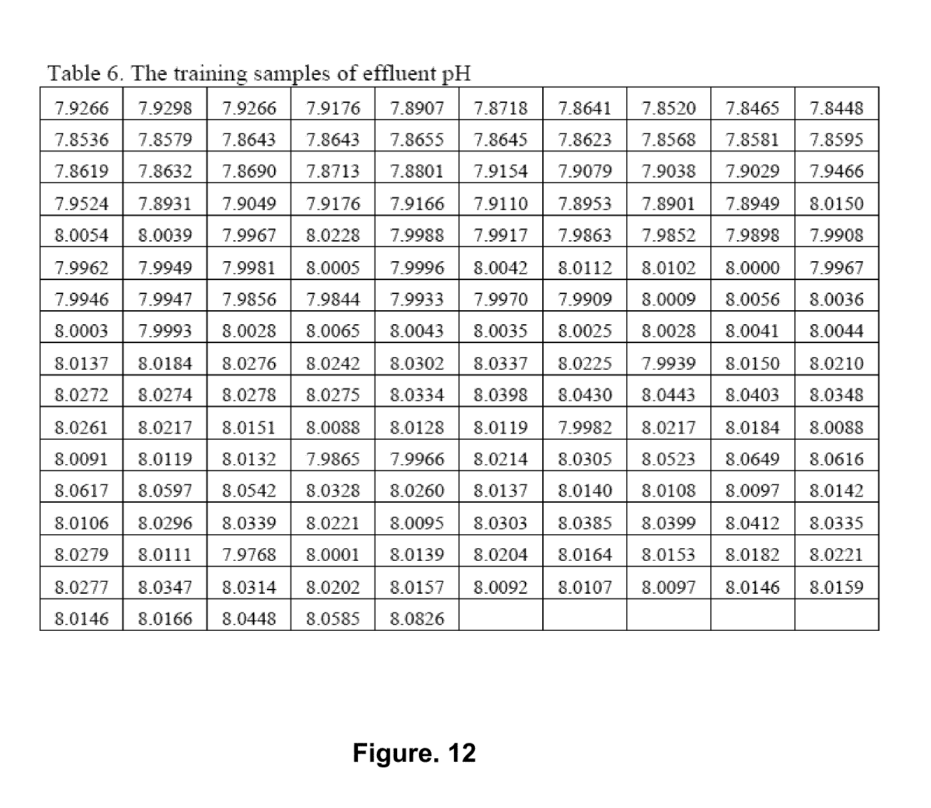

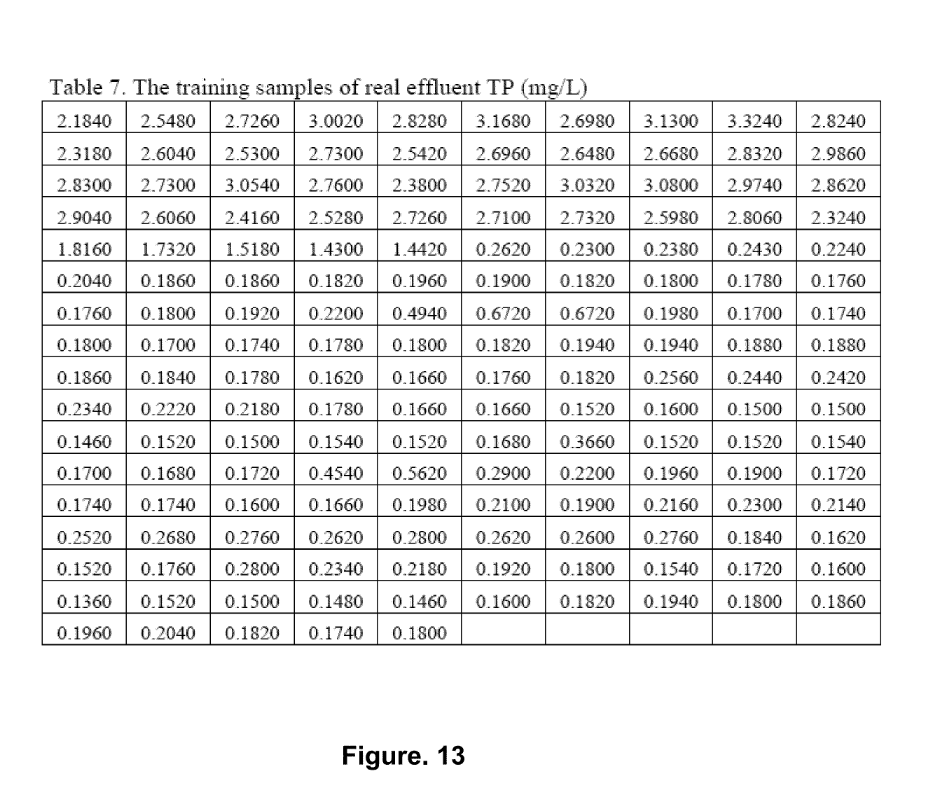

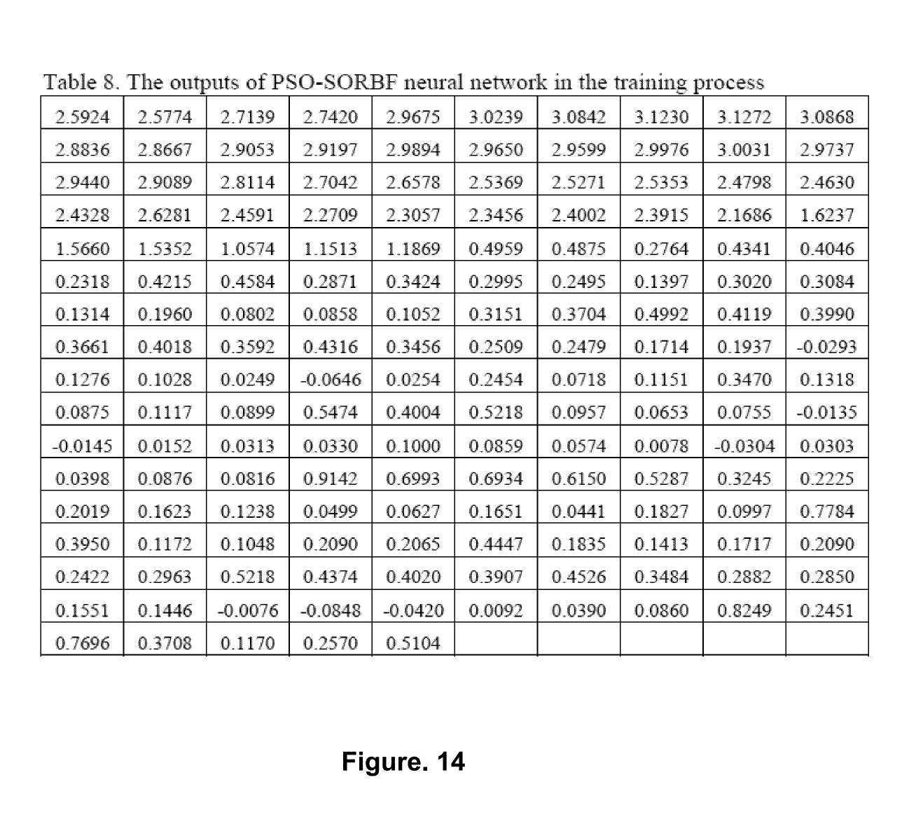

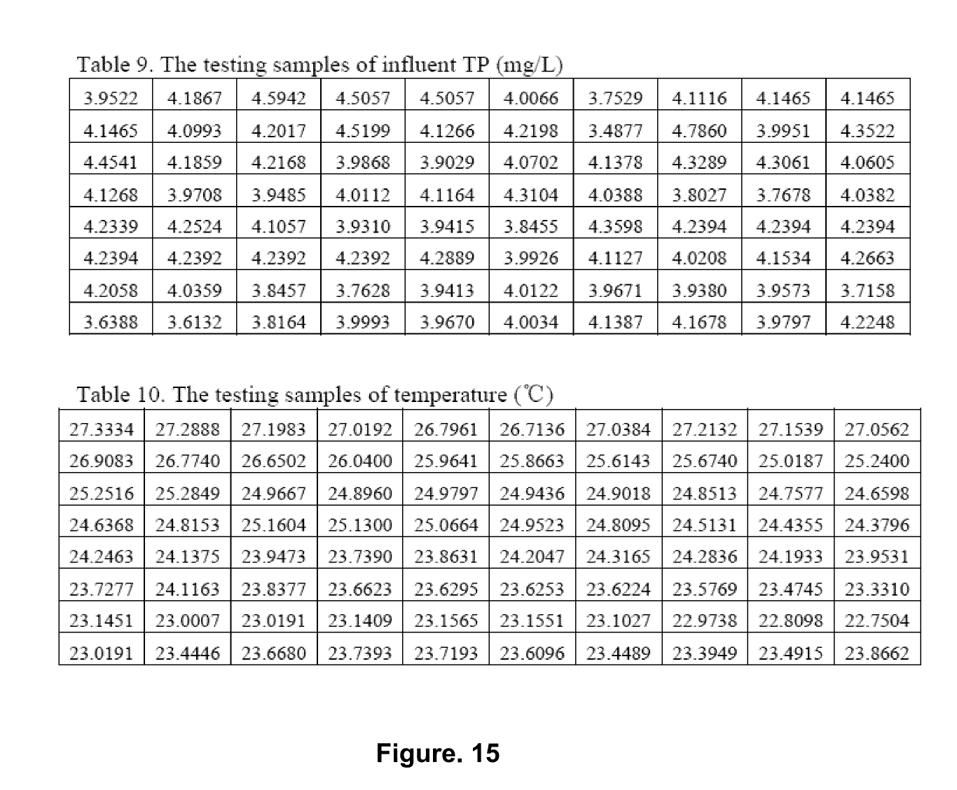

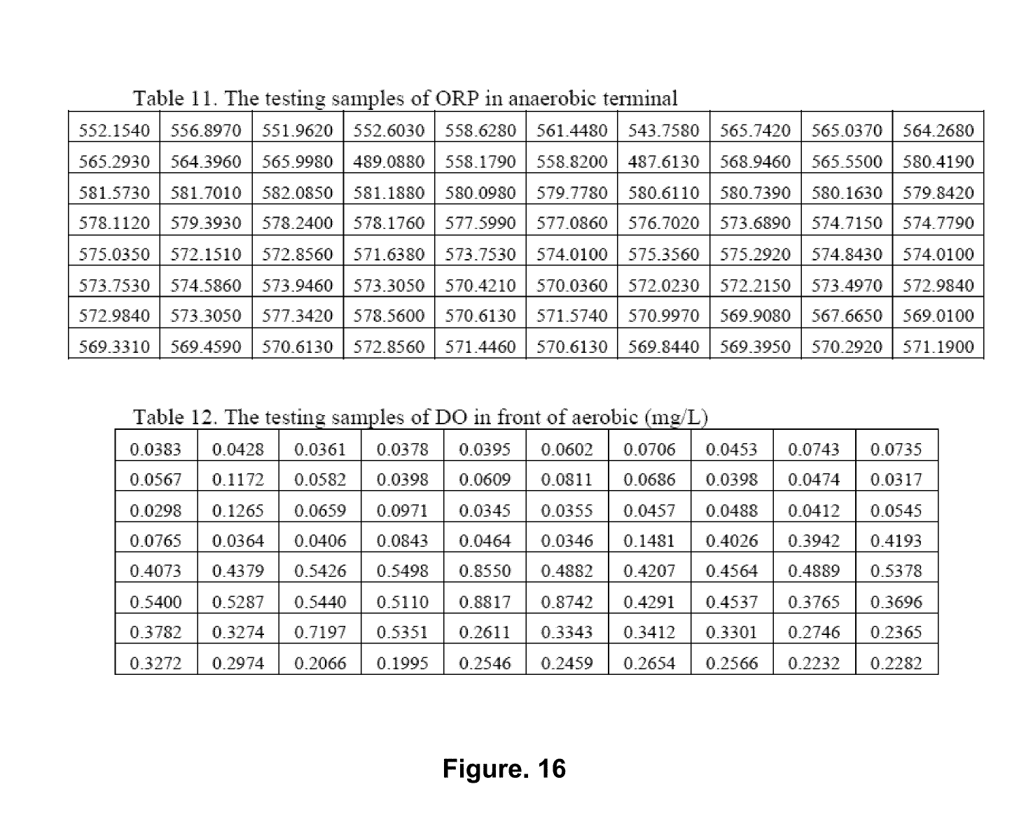

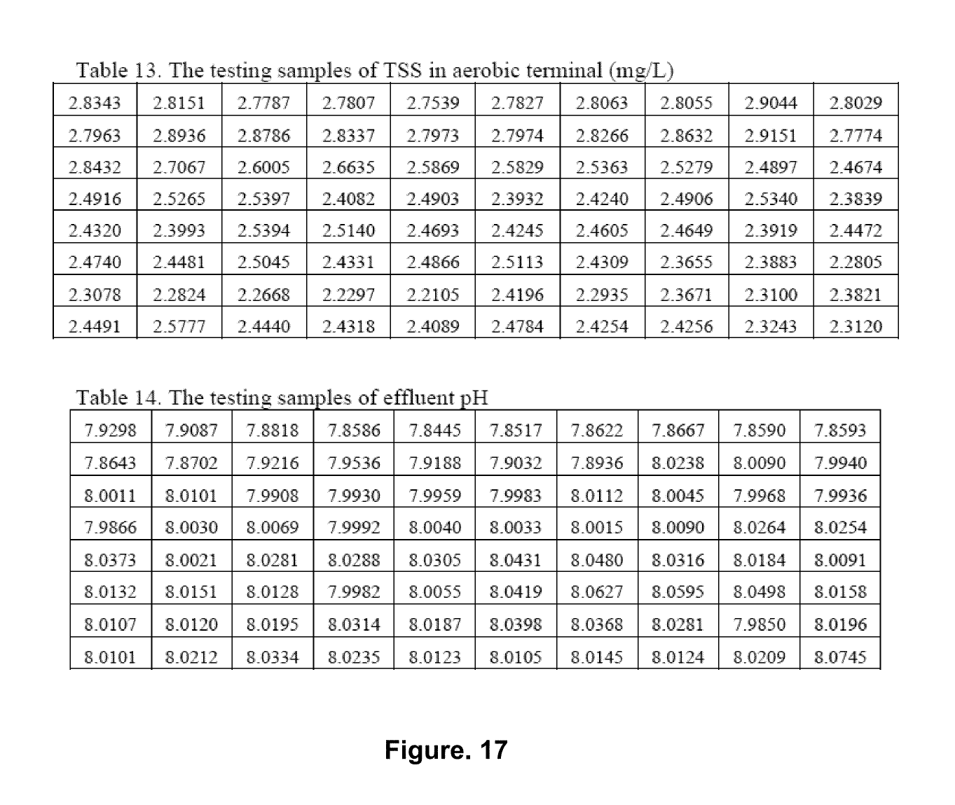

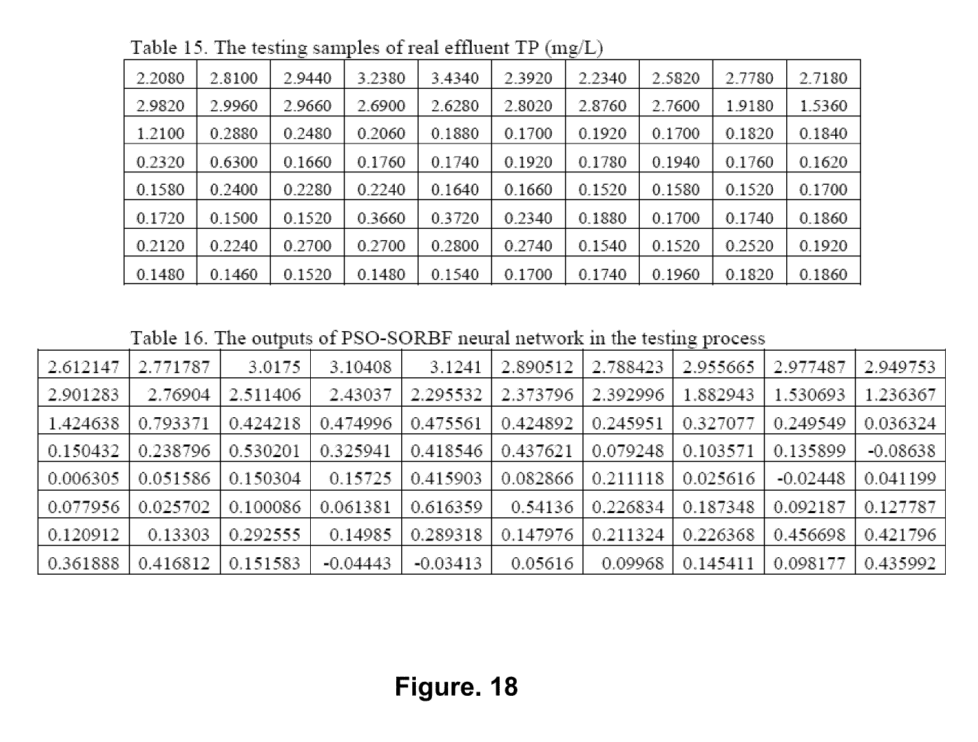

FIGS. 7-18 show Tables 1-16 including experimental data of various implementations. Tables 1-16 show the experimental data in various implementations. Tables 1-7 show the training samples of influent TP, ORP, DO, temperature, TSS, effluent pH and real effluent TP concentration. Table 8 shows the outputs of the PSO-SORBF neural network in the training process. Tables 9-15 show the testing samples of influent TP, ORP, DO, temperature, TSS, effluent pH and real effluent TP concentration. Table 16 shows the outputs of the PSO-SORBF neural network in the predicting process. Moreover, the samples are imported as the sequence from the tables. The first data is in the first row and the first column. Then, the second data is in the first row and the second column. Until all of data is imported from the first row, the data in the second row and following rows are inputted as the same way.

What is claimed is:

1. A method for determining a concentration of total phosphorus (TP) effluent of wastewater in an aerobic tank, the method comprising:
determining, by one or more processors of a computing device, input variables related to the concentration of the TP effluent, the input variables comprising at least one of a centration of TP influent of the wastewater, an oxidation-reduction potential (ORP) in the aerobic tank, a dissolved oxygen (DO) concentration in the aerobic tank, a temperature in the aerobic tank, a total suspended solids (TSS) concentration in the aerobic tank, or a PH value of the TP effluent;
generating, by the one or more processors, a particle swarm optimization self-organizing radial basis function (PSO-SORBF) neural network for TP effluent determination, the PSO-SORBF neural network comprising an input layer, a hidden layer and an output layer;
training, by the one or more processors, the PSO-SORBF neural network using training samples containing sample data of the input variables; and
determining, by the one or more processors, the concentration of the TP effluent using the trained PSO-SORBF neural network.

2. The method of claim 1, wherein the input variables comprise the centration of TP influent of the wastewater, the ORP in the anaerobic tank, the DO concentration in the aerobic tank, the temperature in the aerobic tank, the TSS concentration in the aerobic tank, and the PH value of the TP effluent.

3. The method of claim 2, wherein the generating the PSO-SORBF neural network comprises:
initializing the PSO-SORBF neural network such that the input layer includes six neurons, the hidden layer includes K neurons, and the output layer includes one neuron, K being a positive integer; and
assigning values to parameters of the PSO-SORBF neural network such that:
an input vector of PSO-SORBF neural network at time t is represented by x(t) and is determined using Equation 1:
x(t)=[x₁(t), x₂(t), x₃(t), x₄(t), x₅(t), x₆(t)], (Equation 1)
a number of the training samples is T,
each of x₁(t), x₂(t), x₃(t), x₄(t), x₅(t), and x₆(t) comprises a value of the determined input variables at the time t,
an output of PSO-SORBF neural network is represented by y(t) and determined using Equation 2:
$$y ( t ) = {\sum }_{k = 1}^{K} {w}_{k} ( t ) {φ}_{k} ( x ( t ) ) , \qquad ( Equation 2 )$$
wₖ is an output weight between a k^{th} hidden neuron and an output neuron,
K is a number of hidden neurons,
φₖ is a RBF of k^{th} hidden neuron which is determined by Equation 3:
φₖ(x(t))=e^{(-∥x(t)-μ}^{ₖ}^{(t)∥}^{²}^{/2σ}^{ₖ}^{²}₍ₜ₎₎, (Equation 3)
μₖ denotes a center vector of the k^{th} hidden neuron,
σₖ is a width of the k^{th} hidden neuron, and
an Euclidean distance between x(t) and μₖ(t) is determined using Equation 4:
∥x(t)−μₖ(t)∥. (Equation 4)

4. The method of claim 3, wherein the training the PSO-SORBF neural network comprises:
initializing acceleration constants c₁ and c₂, and a balance factor α such that a position of an i^{th} particle in a searching space is represented aᵢ and determined using Equation 5:
aᵢ=[μ_{i,1}, σ_{i,1}, w_{i,1}, μ_{i,2}, σ_{i,2}, w_{i,2} . . . μ_{i,K}_{ᵢ}, σ_{i,K}_{ᵢ}, w_{i,K}_{ᵢ}], (Equation 5)
wherein:
i an integrate between 1 and s that is a total number of particles,
μ_{i,k}, σ_{i,k}, w_{i,k} are a center, a width and an output weight of the k^{th} hidden neuron in the i^{th} particle, respectively, and
Kᵢ is a number of hidden neurons in the i^{th} particle.

5. The method of claim 4, further comprising:
initializing a velocity of the particle using Equation 6:
vᵢ=[v_{i,1}, v_{i,2}, . . . v_{i,D}_{ᵢ}], (Equation 6)
wherein vᵢ is velocity of i^{th} particle, Dᵢ is a dimension of the ith particle, and Dᵢ equals to 3Kᵢ.

6. The method of claim 5, wherein a fitness value of each particle is determined using Equations 7 and 8 based on an input of neural network x(t) and the dimensions Dᵢ of each particle:
$$\begin{matrix}f ( {a}_{i} ( t ) ) = {E}_{i} ( t ) + α {K}_{i} ( t ) , & ( Equation 7 ) \\ {E}_{i} ( t ) = \sqrt{\frac{1}{2 T} {\sum }_{t = 1}^{T} {( y ( t ) - {y}_{d} ( t ) )}^{2}} , & ( Equation 8 )\end{matrix}$$
wherein:
Kᵢ(t) is a number of hidden neurons in the i^{th} particle at the time t,
T is the number of the training samples,
y_{d}(t) is an expected value of the centration of TP influent at the time t.

7. The method of claim 6, further comprising:
calculating an inertia weight of each particle using Equations 9, 10, 11, and 12:
ωᵢ(t)=γ(t)Aᵢ(t), (Equation 9)
γ(t)=(C−S(t)/1000)^{-t}, (Equation 10)
S(t)=fₘᵢₙ(a(t))/fₘₐₓ(a(t)), (Equation 11)
Aᵢ(t)=f(g(t))/f(aᵢ(t)), (Equation 12)
wherein:
ωᵢ(t) is an inertia weight of the i^{th} particle at the time t, and
C is a constant,
fₘₐₓ(a(t)) are a minimum fitness value and a maximum fitness value at the time t, and
g(t) is a global best position,
fₘᵢₙ(a(t)), fₘₐₓ(a(t)) and g(t) are determined using Equation 13:
$$\{ \begin{matrix}{f}_{min} ( a ( t ) ) = Min ( f ( {a}_{i} ( t ) ) ) \\ {f}_{max} ( a ( t ) ) = Max ( f ( {a}_{i} ( t ) ) )\end{matrix} , \\ g ( t ) = {arg min}_{{p}_{i}} ( f ( {p}_{i} ( t ) ) ) , 1 \leq i \leq s , \qquad ( Equation 13 )$$
wherein pᵢ(t) is a best position of the i^{th} particle.

8. The method of claim 7, further comprising:
updating the position and velocity of each particle according to Equations 14 and 15:
$$\begin{matrix}{v}_{i , d} ( t + 1 ) = ω {v}_{i , d} ( t ) + {c}_{1} {r}_{1} ( {p}_{i , d} ( t ) - {a}_{i , d} ( t ) ) + {c}_{2} {r}_{2} ( {g}_{d} ( t ) - {a}_{i , d} ( t ) ) , & ( Equation 14 ) \\ g ( t ) = {arg min}_{{p}_{i}} ( f ( {p}_{i} ( t ) ) ) , 1 \leq i \leq s , & ( Equation 15 )\end{matrix}$$
wherein r₁ and r₂ are a coefficient of the particle and global best position respectively.

9. The method of claim 8, further comprising:
searching a best number of hidden neurons K_{best} according to the global best position g(t); and
updating the number of hidden neurons in the particles according to Equation 16:
$${K}_{i} = \{ \begin{matrix}{K}_{i} - 1 & if ( {K}_{best} < {K}_{i} ) \\ {K}_{i} + 1 & if ( {K}_{best} \geq {K}_{i} )\end{matrix} . \qquad ( Equation 16 )$$

10. The method of claim 9, further comprising:
importing a training sample x(t+1); and
stop training processes after all of the training samples are imported to the PSO-SORBF neural network.
